(19) United States
(12) Patent Application Publication
(10) Pub. No.: US 2021/0097406 A1
(43) Pub. Date: Apr. 1, 2021

(54) Identification of Clinical Inference Rules

(71) Applicant: International Business Machines Corporation, Armonk, NY (US)

(72) Inventors: Mario J. Lorenzo, Miami, FL (US); Jennifer L. La Rocca, Cary, NC (US); Rebecca L. Dahlman, Rochester, MN (US); Joshua M. Lee, Rochester, MN (US); Kristin E. McNeil, Charlotte, NC (US)

(21) Appl. No.: 16/589,374

(22) Filed: Oct. 1, 2019

(51) Int. Cl. G06N 5/02; G16H 10/60; G16H 50/20; G16H 20/10; G06F 17/27

(52) U.S. Cl. CPC G06N 5/025; G16H 10/60; G06F 17/2705; G16H 20/10; G16H 50/20

(57) ABSTRACT

An inference rules identification mechanism is provided for automatically identifying inference rules. The mechanism parses content of at least one natural language document in a collection of natural language documents utilizing natural language processing to identify a set of attributes and corresponding values present in the content of the at least one natural language document thereby forming a set of attribute/value pairs. For each attribute/value pair in the set of attribute/value pairs, the mechanism determines an affinity correspondence measure of the attribute/value pair with each other attribute/value pair in the set of attribute/value pairs. The mechanism determines, based on the affinity correspondence measures of each attribute/value pair with each other attribute/value pair in the set of attribute/value pairs, a set of inferred rules. The mechanism then automatically generates the inferred rules as rule data structures that are implemented in a cognitive computing model of the cognitive computing system.

### BACKGROUND

The present application relates generally to an improved data processing apparatus and method and more specifically to mechanisms for automatically identifying clinical inference rules.

Each new clinical decision support solution has a specific use case which results in a unique cognitive model. Each customer's use case involves identifying useful information from unstructured text and each customer typically has a massive number of documents, from which the customer usually wants to extract relationships which are the basic conditional rules. Thus, the unique cognitive model varies from use case to use case.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method is provided, in a data processing system comprising a processor and a memory, the memory comprising instructions that are executed by the processor to configure the processor to implement an inference rules identification mechanism for automatically identifying inference rules. The illustrative embodiment parses a content of at least one natural language document in a collection of natural language documents utilizing natural language processing to identify a set of attributes and corresponding values present in the content of the at least one natural language document thereby forming a set of attribute/value pairs. For each attribute/value pair in the set of attribute/value pairs, the illustrative embodiment determines an affinity correspondence measure of the attribute/value pair with each other attribute/value pair in the set of attribute/value pairs. In the illustrative embodiment, the affinity correspondence measure indicates an affinity of one attribute/value pair to another attribute/value pair in the set of attribute/value pairs. The illustrative embodiment determines, based on the affinity correspondence measures of each attribute/value pair with each other attribute/value pair in the set of attribute/value pairs, a set of inferred rules. In the illustrative embodiment, each inferred rule in the set of inferred rules indicates a relationship between the attribute/value pair and a corresponding attribute/value pair. The illustrative embodiment automatically generates the inferred rules as rule data structures that are implemented in a cognitive computing model of the cognitive computing system.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 is an example block diagram illustrating components of a cognitive computing system comprising a clinical inference rules identification mechanism for automatically identifying clinical inference rules in accordance with one illustrative embodiment;

FIG. 2 depicts one example of a set of attributes and their respective values for a set of patients in accordance with an illustrative embodiment:

FIG. 3 depicts an exemplary attribute enumeration chart of all possible attribute combinations in accordance with an illustrative embodiment;

FIG. 4 depicts an exemplary attribute enumeration chart of all remaining attribute combinations once coincidence attribute combinations have been removed in accordance with an illustrative embodiment;

FIG. 5 depicts a schematic diagram of one illustrative embodiment of a cognitive computing system in a computer network;

FIG. 6 is a block diagram of an example data processing system in which aspects of the illustrative embodiments are implemented; and

FIGS. 7A and 7B depict an exemplary flowchart outlining example operations performed by a cognitive computing system implementing a clinical inference rules identification mechanism for automatically identifying clinical inference rules in accordance with one illustrative embodiment.

### DETAILED DESCRIPTION

As noted previously, each new clinical decision support solution has a specific use case which results in a unique cognitive model. Each customer's use case involves identifying useful information from unstructured text and each customer typically has a massive number of documents, from which the customer usually wants to extract relationships which are the basic conditional rules. Thus, the unique cognitive model varies from use case to use case.

Currently, when useful information is identified from the unstructured text, clinical attributes are built based on the concepts and inference rules are built from these attributes. However, it is difficult for a domain expert or engineer to identify all of the attributes that the unstructured text infers. By automatically suggesting inferencing rules based on the customer's unstructured data, the illustrative embodiments reduce the time for customers to build meaningful artificial intelligence models and discover hidden relationships.

Therefore, the illustrative embodiments provide mechanisms to identify attributes and corresponding values that may derive another attribute. From unstructured text, the attributes are extracted for each patient in order to give the unstructured data a level of structure. Each attribute and its value are compared to all other attributes in an attribute set of one patient to identify affinity correspondence. If a rejection threshold is met based on a statistical significance, then the attribute combination is removed from further analysis. This is repeated for each patient and a frequency count is tracked. The attribute/value pairs are evaluated to discover their affinity correspondence and generate a set of inferred rules. The inferred rules are auto-generated based on an attribute model and the patient's unstructured data corpus. These inferred rules are then presented to an end user and the end user may either accept or reject them for use identifying affinity correspondence in other patients' unstructured text.

Before beginning the discussion of the various aspects of the illustrative embodiments in more detail, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on general purpose hardware, software instructions stored on a medium such that the instructions are readily executable by specialized or general purpose hardware, a procedure or method for executing the functions, or a combination of any of the above.

The present description and claims may make use of the terms “a,” “at least one of,” and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein with regard to describing embodiments and features of the invention, is not intended to be limiting of any particular implementation for accomplishing and/or performing the actions, steps, processes, etc., attributable to and/or performed by the engine. An engine may be, but is not limited to, software, hardware and/or firmware or any combination thereof that performs the specified functions including, but not limited to, any use of a general and/or specialized processor in combination with appropriate software loaded or stored in a machine readable memory and executed by the processor. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

As noted above, the illustrative embodiments of the present invention provides a methodology, apparatus, system and computer program product for automatically identifying clinical inference rules. The following illustrates the operations of a cognitive computing system in which a clinical inference rules identification mechanism automatically identifies clinical inference rules. The clinical inference rules identification mechanism extracts, from unstructured text of a set of patients, attributes and corresponding values for each patient in order to give the unstructured data a level of structure. The clinical inference rules identification mechanism compares each attribute and its value to all other attributes in an attribute set of one patient to identify affinity correspondence. If the clinical inference rules identification mechanism determines that a rejection threshold is met based on a statistical significance, then the clinical inference rules identification mechanism removes the attribute combination from further analysis. The clinical inference rules identification mechanism repeats this process for each patient and tracks a frequency count. The clinical inference rules identification mechanism then evaluates the attribute/value pairs to discover their affinity correspondence and generate a set of inferred rules. The clinical inference rules identification mechanism auto-generates inferred rules based on an attribute model and the patient's unstructured data corpus. The clinical inference rules identification mechanism then presents the set of inferred rules to an end user at which time the end user may either accept or reject one or more of the set of inferred rules and utilize the remaining inferred rules to identify affinity correspondence in other patients' unstructured text.

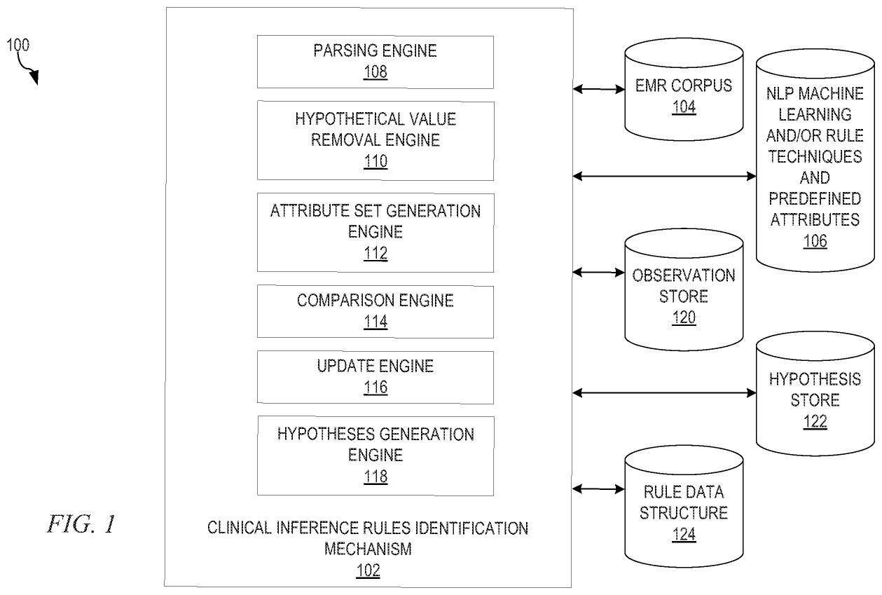

FIG. 1 is an example block diagram illustrating components of a cognitive computing system comprising a clinical inference rules identification mechanism for automatically identifying clinical inference rules in accordance with one illustrative embodiment. As shown in FIG. 1, cognitive computing system 100 comprises clinical inference rules identification mechanism 102, electronic medical records (EMR) corpus 104, and Natural Language Processing (NLP) machine learning and/or rule techniques and predefined attributes 106. Clinical inference rules identification mechanism 102 further comprises parsing engine 108, hypothetical value removal engine 110, attribute set generation engine 112, comparison engine 114, update engine 116, and hypotheses generation engine 118.

In order to automatically identify clinical inference rules, parsing engine 108 parses a set of unstructured medical records (natural language documents) in EMR corpus 104 using a set of NLP machine learning and/or rule techniques and predefined attributes in NLP machine learning and/or rule techniques and predefined attributes 106. By parsing the set of medical records, parsing engine 108 generates a set of annotations, lexical terms, attributes values, or the like. For example, by parsing the following clinical unstructured text—“Stage IV adenocarcinoma of the right lung, with multiple bilateral pulmonary nodules, and tumor is M1 stage”—parsing engine 108 may identify, as one example, the attribute “Stage=IV.”

Utilizing the generated set of annotations, lexical terms, attributes values, or the like, hypothetical value removal engine 110 removes annotations, lexical terms, attributes values, or the like, values that are hypothetical. For example, if a clinical unstructured text from which an annotation, lexical term, attributes value, or the like, was parsed reads—“If patient has stage IV cancer, then we will proceed with chemotherapy”—the unstructured text indicates that the patient might have cancer but it does not identify the patient as actually having cancer. As a result, the hypothetical value removal engine 110 removes the annotation, lexical term, attributes value, or the like, found for this unstructured text so that the associated annotations, lexical terms, attributes values, or the like, do not negatively impact the resulting inference rule(s).

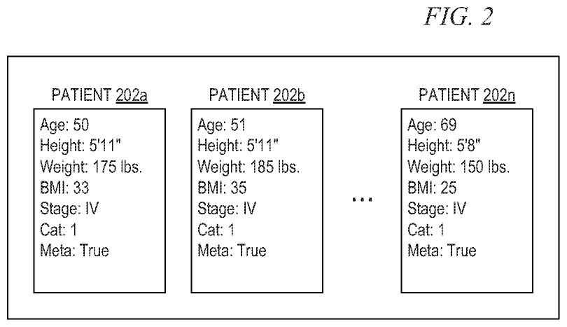

With the remaining set of annotations, lexical terms, attributes values, or the like, attribute set generation engine 112 generates a list of attribute sets. Each attribute set comprises a list of attributes with their corresponding values. For example, as is illustrated in FIG. 2, for each of patients 202a-202n, the attributes of: Age, Body Mass Index (BMI), Stage, cM Category (Cat), and Metastatic (Meta) Category, have been identified, as well as their respective values in accordance with an illustrative embodiment. For each attribute in the list of attributes, attribute set generation engine 112 generate a finding count of each attribute that occurs within the set of unstructured medical records in EMR corpus 104. For example, if the set of unstructured medical records in EMR corpus 104 only produces the output shown in FIG. 2, then Age occurs 3 times and 3 would be used as the total Age finding count.

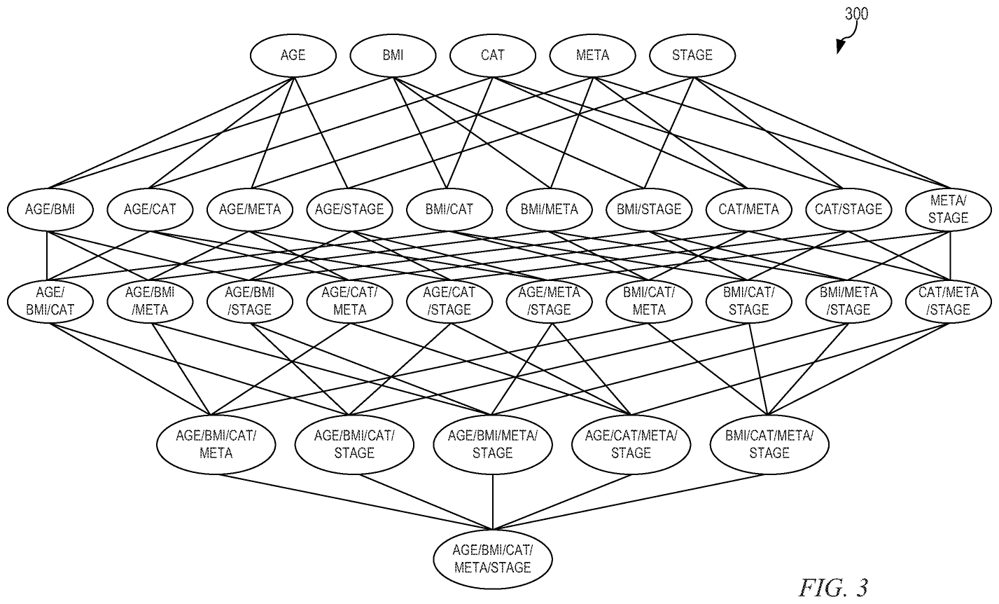

Comparison engine 114 obtains one attribute finding in the list of attributes and determines an affinity correspondence measure of the attribute and its corresponding value with each other attribute and its corresponding value in the set of attributes and corresponding values. That is, comparison engine 114 compares each attribute to each of the other attributes within the list of attributes in order to enumerate attribute value pairs to other attribute value pairs. For example, for the attributes of: Age, Body Mass Index (BMI), Stage, cM Category (Cat), and Metastatic (Meta) Category; one attribute is compared across the other attributes in the attribute set to generate an attribute enumeration table of:
Stage->Age
Stage->BMI
Stage->Cat
Stage->Meta
   Comparison engine 114 repeats this operation for other attributes in the list of attributes that have not been compared within in the list of attributes. Once comparison engine 114 completes all comparisons for all attributes in the list of attributes, comparison engine 114 generates an attribute enumeration chart of all possible attribute combinations such as that illustrated in FIG. 3 in accordance with an illustrative embodiment. As is shown, attribute enumeration chart 300 illustrates all possible attribute combinations of the attributes: Age, BMI, Cat, Meta, and Stage to be analyzed by comparison engine 114. As comparison engine 114 identifies each attribute and attribute comparison within the unstructured text on patient-by-patient basis, comparison engine 114 increases frequency counter for the attribute and attribute comparison, which generates an attribute pair finding frequency number. Thus, as one example, in a comparison of the compared attributes of patient 202a and patient 202b in FIG. 2, comparison engine 114 would identify a commonality that both patient 202a and patient 202b have a Height of 5′11″ and Meta equal to “True” in the two attribute sets as well as a Stage of “IV”, a Cat of “1”, and a Meta of “True”.

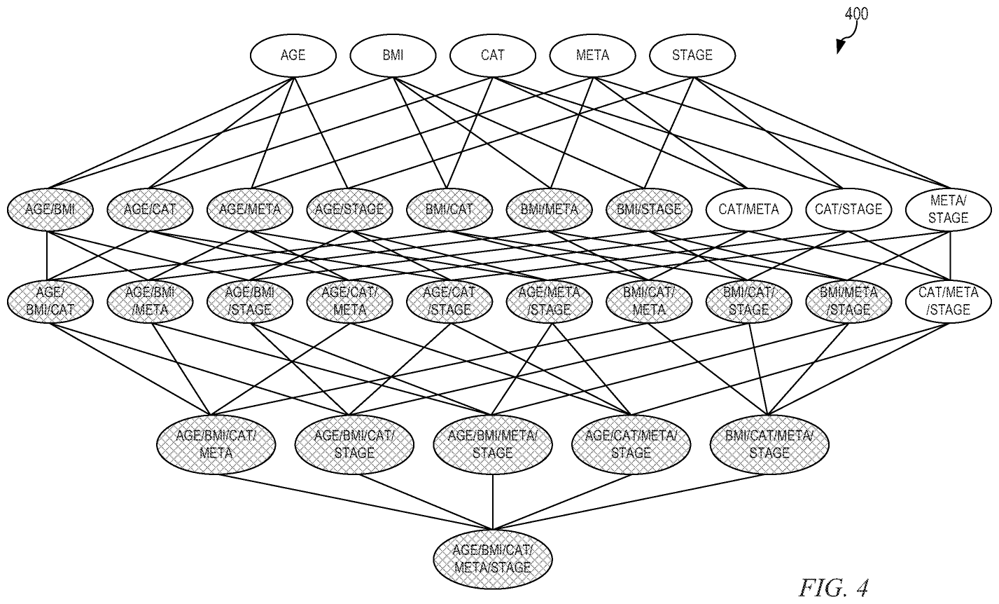

Once two attributes sets for two patients have been processed and as each additional attribute set for an additional patient is processed, comparison engine 114 determines whether a rejection threshold has been met for the attribute combination, such as, for example, a frequency of the combination is >0.05 which means high variance and/or no correlation. If comparison engine 114 determines that the rejection threshold has been met, then comparison engine 114 discontinues analysis of that attribute combination for any further attribute sets of additional patients and moves the attribute combination to a coincidence list. As each additional attribute set of another patient is analyzed by comparison engine 114, comparison engine 114 continuously removes attribute pair combinations based on the rejection threshold and thus, reduces a number of attribute pairs under consideration, which decreases computational processing time. Thus, once comparison engine 114 has processed patients 202a-202n, as is illustrated in the attribute enumeration chart of all remaining attribute combinations in FIG. 4 in accordance with an illustrative embodiment, all attribute combinations that are illustrated in “crosshatch” have met the rejection threshold since there was no correlation and thus, comparison engine 114 removed those attribute combinations from further comparison analysis. Therefore, in reviewing further attribute sets for other patients, comparison engine 114 would only compare the four remaining attribute combinations that do not meet the rejection threshold.

Once all attribute sets for all of patients 202a-202n have been analyzed, update engine 116 updates observation store 120 with the observation finding, i.e. common attributes of a Stage of “IV”, a Cat of “1”, and a Meta of “True”, indicating that these attributes have not met the rejection threshold and instead have met an acceptance threshold, such as, for example, a frequency of the combination is <=0.05 which means low variance and/or 95% correspondence as well as those attributes that failed to meet the rejection threshold and were added to the coincidence list. Once update engine has updated observation store 120 with the observation finding, hypotheses generation engine 118 retrieves the observation finding from observation store 120 and generates a hypothesis list comprising a hypothesized piece of evidence, i.e. an inferred rule, which identifies either a correlation or a coincidence between two or more attributes identified in the observation findings. For example, utilizing a concordance correlation coefficient statistical measurement, hypotheses generation engine 118 is able to identify a measure of agreement between two attributes. For example, with regard to the patients and their associated attributes in FIG. 2, the stage attribute being equal to IV is found in 95% of patients with cM Category (Cat) attribute equal to 1. Further, the stage attribute equal to IV is found only in 1% of patients with a BMI of 50 so this indicates that the association is coincidence. Hypotheses generation engine 118 and adds each hypothesized piece of information to a hypothesis store 122. Hypotheses generation engine 118 the automatically generates a set of inferred rules based on each hypothesized piece of information as a rule data structures 124, where the set of inferred rules are then implemented in a cognitive computing model of cognitive computing system 100 to process other natural language documents.

Additionally, hypotheses generation engine 118 may discard any attributes that correlate with too many other attributes. For example, hypotheses generation engine 118 may apply a statistical error where, when a contradiction is identified based on the statistical error, the hypothesized piece of information may be marked as invalid or removed from hypothesis store 122.

Thus, cognitive computing system 100 is specifically tailored to identify attributes and corresponding values that may derive another attribute. From unstructured text, the attributes are extracted for each patient in order to give the unstructured data a level of structure. Each attribute and its value are compared to all other attributes in an attribute set of one patient to identify affinity correspondence. If a rejection threshold is met based on a statistical significance, then the attribute combination is removed from further analysis. This is repeated for each patient and a frequency count is tracked. The attribute/value pairs are evaluated to discover their affinity correspondence and generate a set of inferred rules. The inferred rules are auto-generated based on an attribute model and the patient's unstructured data corpus. These inferred rules are then presented to an end user and the end user may either accept or reject them for use identifying affinity correspondence in other patients' unstructured text.

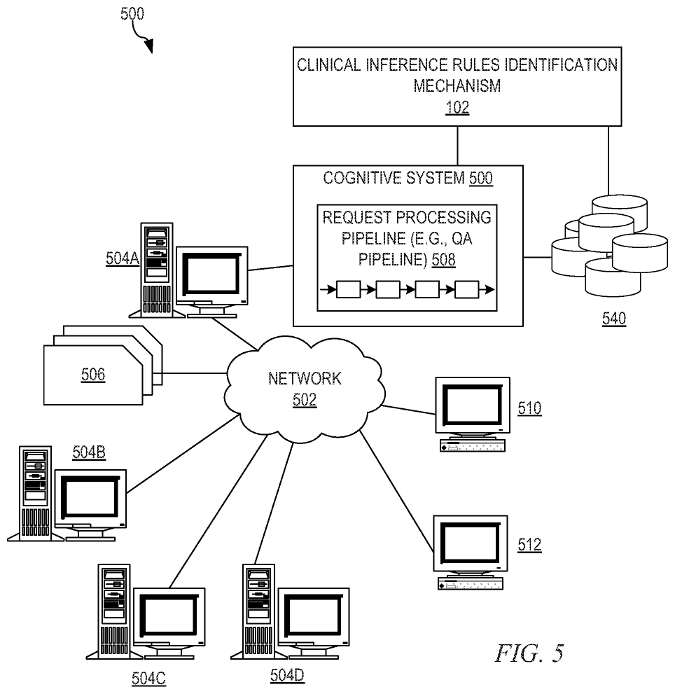

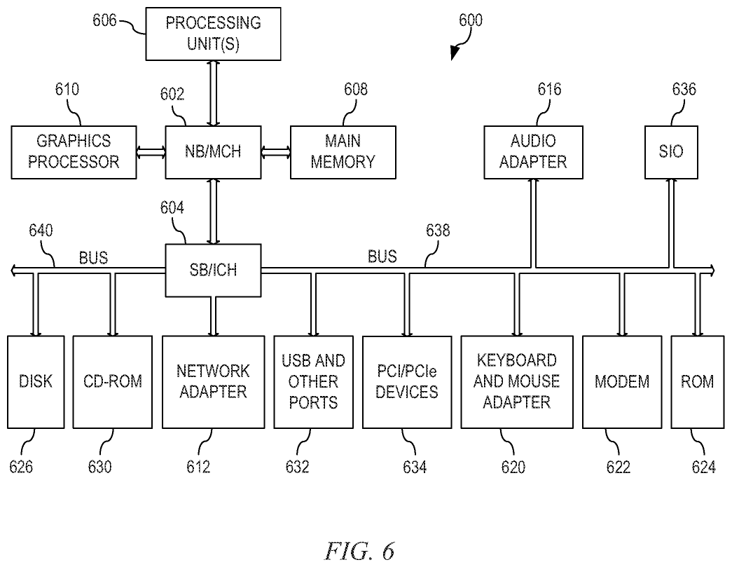

It is clear from the above, that the illustrative embodiments may be utilized in many different types of data processing environments. In order to provide a context for the description of the specific elements and functionality of the illustrative embodiments, FIGS. 5-6 are provided hereafter as example environments in which aspects of the illustrative embodiments may be implemented. It should be appreciated that FIGS. 5-6 are only examples and are not intended to assert or imply any limitation with regard to the environments in which aspects or embodiments of the present invention may be implemented. Many modifications to the depicted environments may be made without departing from the spirit and scope of the present invention.

FIGS. 5-6 are directed to describing an example cognitive computing system that implements a clinical inference rules identification mechanism for automatically identifying clinical inference rules. Therefore, the clinical inference rules identification mechanism identifies attributes and corresponding values that may derive another attribute. From unstructured text, the clinical inference rules identification mechanism extracts attributes for each patient in order to give the unstructured data a level of structure. The clinical inference rules identification mechanism compares each attribute and its value to all other attributes in an attribute set of one patient to identify affinity correspondence. If a rejection threshold is met based on a statistical significance, then the clinical inference rules identification mechanism removes the attribute combination from further analysis. The clinical inference rules identification mechanism repeats this process for each patient and a frequency count is tracked. The clinical inference rules identification mechanism evaluates the attribute/value pairs to discover their affinity correspondence and generate a set of inferred rules. The clinical inference rules identification mechanism automatically generates a set of inferred rules based on an attribute model and the patient's unstructured data corpus. The clinical inference rules identification mechanism then presents the set of inferred rules to an end user and the end user may either accept or reject them for use identifying affinity correspondence in other patients' unstructured text.

It should be appreciated that the cognitive computing system and clinical inference rules identification mechanism, while shown as having a single request processing pipeline in the examples hereafter, may in fact have multiple request processing pipelines. Each request processing pipeline may be separately trained and/or configured to process requests associated with different domains or be configured to perform the same or different analysis on input requests, depending on the desired implementation. For example, in some cases, a request processing pipeline may be trained to extract attributes for each patient in order to give the unstructured data a level of structure. As another example, a different request processing pipeline may be configured to compare each attribute and its value to all other attributes in an attribute set of one patient to identify affinity correspondence. As still a further example, an even different request processing pipeline may be configured to evaluate the attribute/value pairs to discover their affinity correspondence and generate a set of inferred rules.

Moreover, each request processing pipeline may have its own associated corpus or corpora that they ingest and operate on, e.g., one corpus for patients' medical information, another corpus for medical conditions, or the like, in the above examples. In some cases, the request processing pipelines may each operate on the same domain of requests but may have different configurations, e.g., different annotators or differently trained annotators, such that different analysis and potential responses are generated. The cognitive computing system may provide additional logic for routing requests to the appropriate request processing pipeline, such as based on a determined domain of the input request, combining and evaluating final results generated by the processing performed by multiple request processing pipelines, and other control and interaction logic that facilitates the utilization of multiple request processing pipelines.

It should be appreciated that while the present invention will be described in the context of the cognitive computing system and clinical inference rules identification mechanism implementing one or more request processing pipelines that operate on a request, the illustrative embodiments are not limited to such. Rather, the mechanisms of the illustrative embodiments may operate on requests that are posed as “questions” or formatted as requests for the cognitive computing system to perform cognitive operations on a specified set of input data using the associated corpus or corpora and the specific configuration information used to configure the cognitive computing system.

As will be discussed in greater detail hereafter, the illustrative embodiments may be integrated in, augment, and extend the functionality of the request processing pipeline with regard to rank search results based on, for example, an affinity correspondence. For example, parsing a content of at least one natural language document in a collection of natural language documents utilizing natural language processing to identify a set of attributes and corresponding values present in the content of the at least one natural language document thereby forming a set of attribute/value pairs; for each attribute/value pair in the set of attribute/value pairs, determining an affinity correspondence measure of the attribute/value pair with each other attribute/value pair in the set of attribute/value pairs, wherein the affinity correspondence measure indicates an affinity of one attribute/value pair to another attribute/value pair in the set of attribute/value pairs; determining, based on the affinity correspondence measures of each attribute/value pair with each other attribute/value pair in the set of attribute/value pairs, a set of inferred rules, wherein each inferred rule in the set of inferred rules indicates a relationship between the attribute/value pair and a corresponding attribute/value pair; and automatically generating the inferred rules as rule data structures that are implemented in a cognitive computing model of the cognitive computing system to process other natural language documents.

It should be appreciated that the mechanisms described in FIGS. 5-6 are only examples and are not intended to state or imply any limitation with regard to the type of cognitive computing system mechanisms with which the illustrative embodiments are implemented. Many modifications to the example cognitive computing system shown in FIGS. 5-6 may be implemented in various embodiments of the present invention without departing from the spirit and scope of the present invention.

As an overview, a cognitive computing system is a specialized computer system, or set of computer systems, configured with hardware and/or software logic (in combination with hardware logic upon which the software executes) to emulate human cognitive functions. These cognitive computing systems apply human-like characteristics to conveying and manipulating ideas which, when combined with the inherent strengths of digital computing, can solve problems with high accuracy and resilience on a large scale. A cognitive computing system performs one or more computer-implemented cognitive operations that approximate a human thought process as well as enable people and machines to interact in a more natural manner so as to extend and magnify human expertise and cognition. A cognitive computing system comprises artificial intelligence logic, such as natural language processing (NLP) based logic, for example, and machine learning logic, which may be provided as specialized hardware, software executed on hardware, or any combination of specialized hardware and software executed on hardware. The logic of the cognitive computing system implements the cognitive operation(s), examples of which include, but are not limited to, question answering, identification of related concepts within different portions of content in a corpus, intelligent search algorithms, such as Internet web page searches.

IBM Watson™ is an example of one such cognitive computing system which can process human readable language and identify inferences between text passages with human-like high accuracy at speeds far faster than human beings and on a larger scale. In general, such cognitive computing systems are able to perform the following functions:
Navigate the complexities of human language and understanding,
Ingest and process vast amounts of structured and unstructured data,
Generate and evaluate hypothesis,
Weigh and evaluate responses that are based only on relevant evidence,
Provide situation-specific advice, insights, and guidance,
Improve knowledge and learn with each iteration and interaction through machine learning processes,
Enable decision making at the point of impact (contextual guidance),
Scale in proportion to the task,
Extend and magnify human expertise and cognition,
Identify resonating, human-like attributes and traits from natural language,
Deduce various language specific or agnostic attributes from natural language,
High degree of relevant recollection from data points (images, text, voice) (memorization and recall),
Predict and sense with situational awareness that mimic human cognition based on experiences, or
Answer questions based on natural language and specific evidence.

In one aspect, cognitive computing systems provide mechanisms for responding to requests posed to these cognitive computing systems using a request processing pipeline and/or process requests which may or may not be posed as natural language requests. The requests processing pipeline is an artificial intelligence application executing on data processing hardware that responds to requests pertaining to a given subject-matter domain presented in natural language. The request processing pipeline receives inputs from various sources including input over a network, a corpus of electronic documents or other data, data from a content creator, information from one or more content users, and other such inputs from other possible sources of input. Data storage devices store the corpus of data. A content creator creates content in a document for use as part of a corpus of data with the request processing pipeline. The document may include any file, text, article, or source of data for use in the requests processing system. For example, a request processing pipeline accesses a body of knowledge about the domain, or subject matter area, e.g., financial domain, medical domain, legal domain, etc., where the body of knowledge (knowledgebase) can be organized in a variety of configurations, e.g., a structured repository of domain-specific information, such as ontologies, or unstructured data related to the domain, or a collection of natural language documents about the domain.

Content users input requests to cognitive computing systems which implements the request processing pipeline. The request processing pipeline then responds to the requests using the content in the corpus of data by evaluating documents, sections of documents, portions of data in the corpus, or the like. When a process evaluates a given section of a document for semantic content, the process can use a variety of conventions to query such document from the request processing pipeline, e.g., sending the query to the request processing pipeline as a well-formed requests which is then interpreted by the request processing pipeline and a response is provided containing one or more responses to the request. Semantic content is content based on the relation between signifiers, such as words, phrases, signs, and symbols, and what they stand for, their denotation, or connotation. In other words, semantic content is content that interprets an expression, such as by using Natural Language Processing.

As will be described in greater detail hereafter, the request processing pipeline receives a request, parses the request to extract the major features of the request, uses the extracted features to formulate queries, and then applies those queries to the corpus of data. Based on the application of the queries to the corpus of data, the request processing pipeline generates a set of responses to the request, by looking across the corpus of data for portions of the corpus of data that have some potential for containing a valuable response to the request. The request processing pipeline then performs deep analysis on the language of the request and the language used in each of the portions of the corpus of data found during the application of the queries using a variety of reasoning algorithms. There may be hundreds or even thousands of reasoning algorithms applied, each of which performs different analysis, e.g., comparisons, natural language analysis, lexical analysis, or the like, and generates a score. For example, some reasoning algorithms may look at the matching of terms and synonyms within the language of the request and the found portions of the corpus of data. Other reasoning algorithms may look at temporal or spatial features in the language, while others may evaluate the source of the portion of the corpus of data and evaluate its veracity.

As mentioned above, request processing pipeline mechanisms operate by accessing information from a corpus of data or information (also referred to as a corpus of content), analyzing it, and then generating answer results based on the analysis of this data. Accessing information from a corpus of data typically includes: a database query that answers requests about what is in a collection of structured records, and a search that delivers a collection of document links in response to a query against a collection of unstructured data (text, markup language, etc.). Conventional request processing systems are capable of generating answers based on the corpus of data and the input request, verifying answers to a collection of request for the corpus of data, correcting errors in digital text using a corpus of data, and selecting responses to requests from a pool of potential answers, i.e. candidate answers.

FIG. 5 depicts a schematic diagram of one illustrative embodiment of a cognitive computing system 500 implementing a request processing pipeline 508, which in some embodiments may be a request processing pipeline, in a network 502. For purposes of the present description, it will be assumed that the request processing pipeline 508 is implemented as a request processing pipeline that operates on structured and/or unstructured requests in the form of input questions. One example of a question processing operation which may be used in conjunction with the principles described herein is described in U.S. Patent Application Publication No. 2011/0125734, which is herein incorporated by reference in its entirety. The cognitive computing system 500 is implemented on one or more computing devices 504A-D (comprising one or more processors and one or more memories, and potentially any other computing device elements generally known in the art including buses, storage devices, communication interfaces, and the like) connected to the network 502. For purposes of illustration only, FIG. 5 depicts the cognitive computing system 500 being implemented on computing device 504A only, but as noted above the cognitive computing system 500 may be distributed across multiple computing devices, such as a plurality of computing devices 504A-D. The network 502 includes multiple computing devices 504A-D, which may operate as server computing devices, and 510-512 which may operate as client computing devices, in communication with each other and with other devices or components via one or more wired and/or wireless data communication links, where each communication link comprises one or more of wires, routers, switches, transmitters, receivers, or the like. In some illustrative embodiments, the cognitive computing system 500 and network 502 enables question processing and answer generation (QA) functionality for one or more cognitive computing system users via their respective computing devices 510-512. In other embodiments, the cognitive computing system 500 and network 502 may provide other types of cognitive operations including, but not limited to, request processing and cognitive response generation which may take many different forms depending upon the desired implementation, e.g., cognitive information retrieval, training/instruction of users, cognitive evaluation of data, or the like. Other embodiments of the cognitive computing system 500 may be used with components, systems, sub-systems, and/or devices other than those that are depicted herein.

The cognitive computing system 500 is configured to implement a request processing pipeline 508 that receive inputs from various sources. The requests may be posed in the form of a natural language question, natural language request for information, natural language request for the performance of a cognitive operation, or the like. For example, the cognitive computing system 500 receives input from the network 502, a corpus or corpora of electronic documents 506 or 540, cognitive computing system users, and/or other data and other possible sources of input. In one embodiment, some or all of the inputs to the cognitive computing system 500 are routed through the network 502. The various computing devices 504A-D on the network 502 include access points for content creators and cognitive computing system users. Some of the computing devices 504A-D includes devices for a database storing the corpus or corpora of data 506 or 540 (which is shown as a separate entity in FIG. 5 for illustrative purposes only). Portions of the corpus or corpora of data 506 or 540 may also be provided on one or more other network attached storage devices, in one or more databases, or other computing devices not explicitly shown in FIG. 5. The network 502 includes local network connections and remote connections in various embodiments, such that the cognitive computing system 500 may operate in environments of any size, including local and global, e.g., the Internet.

In one embodiment, the content creator creates content in a document of the corpus or corpora of data 506 or 540 for use as part of a corpus of data with the cognitive computing system 500. The document includes any file, text, article, or source of data for use in the cognitive computing system 500. Cognitive computing system users access the cognitive computing system 500 via a network connection or an Internet connection to the network 502, and requests to the cognitive computing system 500 that are responded to/processed based on the content in the corpus or corpora of data 506 or 540. In one embodiment, the requests are formed using natural language. The cognitive computing system 500 parses and interprets the request via a pipeline 508, and provides a response to the cognitive computing system user, e.g., cognitive computing system user 510, containing one or more responses to the request posed, response to the request, results of processing the request, or the like. In some embodiments, the cognitive computing system 500 provides a response to users in a ranked list of candidate answers/responses while in other illustrative embodiments, the cognitive computing system 500 provides a single final response or a combination of a response and ranked listing of other candidate responses.

The cognitive computing system 500 implements the pipeline 508 which comprises a plurality of stages for processing a request based on information obtained from the corpus or corpora of data 506 or 540. The pipeline 508 generates responses for the request based on the processing of the request and the corpus or corpora of data 506 or 540.

In some illustrative embodiments, the cognitive computing system 500 may be the IBM Watson™ cognitive system available from International Business Machines Corporation of Armonk, N.Y., which is augmented with the mechanisms of the illustrative embodiments described hereafter. As outlined previously, a pipeline of the IBM Watson™ cognitive system receives a request which it then parses to extract the major features of the request, which in turn are then used to formulate queries that are applied to the corpus or corpora of data 506 or 540. Based on the application of the queries to the corpus or corpora of data 506 or 540, a set of hypotheses, or candidate responses to the request, are generated by looking across the corpus or corpora of data 506 or 540 for portions of the corpus or corpora of data 506 or 540 (hereafter referred to simply as the corpus 506 or 540) that have some potential for containing a valuable response to the response. The pipeline 508 of the IBM Watson™ cognitive system then performs deep analysis on the language of the request and the language used in each of the portions of the corpus 506 or 540 found during the application of the queries using a variety of reasoning algorithms.

The scores obtained from the various reasoning algorithms are then weighted against a statistical model that summarizes a level of confidence that the pipeline 508 of the IBM Watson™ cognitive computing system 500, in this example, has regarding the evidence that the potential candidate answer is inferred by the request. This process is repeated for each of the candidate answers to generate a ranked listing of candidate answers which may then be presented to the user that submitted the request, e.g., a user of client computing device 510, or from which a final response is selected and presented to the user.

As noted above, while the input to the cognitive computing system 500 from a client device may be posed in the form of a natural language request; the illustrative embodiments are not limited to such. Rather, the request may in fact be formatted or structured as any suitable type of request which may be parsed and analyzed using structured and/or unstructured input analysis, including but not limited to the natural language parsing and analysis mechanisms of a cognitive computing system such as IBM Watson™, to determine the basis upon which to perform cognitive analysis and providing a result of the cognitive analysis. In the case of a cognitive computing system, this analysis may involve processing patient medical records, medical guidance documentation from one or more corpora, and the like, to provide a cognitive computing system result. In particular, the mechanisms of the cognitive computing system may process drug-adverse events or adverse drug reaction pairings when performing the cognitive computing system result, e.g., a diagnosis or treatment recommendation.

In the context of the present invention, cognitive computing system 500 may provide a cognitive functionality for automatically identifying clinical inference rules. For example, depending upon the particular implementation, the healthcare based operations may comprise patient diagnostics, medical treatment recommendation systems, personal patient care plan generation and monitoring, patient electronic medical record (EMR) evaluation for various purposes, such as for identifying patients that are suitable for a medical trial or a particular type of medical treatment, or the like. Thus, the cognitive computing system 500 may be a healthcare cognitive computing system 500 that operates in the medical or healthcare type domains and which may process requests for such healthcare operations via the request processing pipeline 508 input as either structured or unstructured requests, natural language input requests, or the like. In one illustrative embodiment, the cognitive computing system 500 is an cognitive computing system that parses a content of at least one natural language document in a collection of natural language documents utilizing natural language processing to identify a set of attributes and corresponding values present in the content of the at least one natural language document thereby forming a set of attribute/value pairs; for each attribute/value pair in the set of attribute/value pairs, determines an affinity correspondence measure of the attribute/value pair with each other attribute/value pair in the set of attribute/value pairs, wherein the affinity correspondence measure indicates an affinity of one attribute/value pair to another attribute/value pair in the set of attribute/value pairs; determines, based on the affinity correspondence measures of each attribute/value pair with each other attribute/value pair in the set of attribute/value pairs, a set of inferred rules, wherein each inferred rule in the set of inferred rules indicates a relationship between the attribute/value pair and a corresponding attribute/value pair; and automatically generates the inferred rules as rule data structures that are implemented in a cognitive computing model of the cognitive computing system to process other natural language documents.

As shown in FIG. 5, the cognitive computing system 500 is further augmented, in accordance with the mechanisms of the illustrative embodiments, to include logic implemented in specialized hardware, software executed on hardware, or any combination of specialized hardware and software executed on hardware, for implementing a cognitive computing system 100 of FIG. 1. As described previously, the cognitive computing system 100 performs an automatic identification of clinical inference rules.

As noted above, the mechanisms of the illustrative embodiments are rooted in the computer technology arts and are implemented using logic present in such computing or data processing systems. These computing or data processing systems are specifically configured, either through hardware, software, or a combination of hardware and software, to implement the various operations described above. As such, FIG. 6 is provided as an example of one type of data processing system in which aspects of the present invention may be implemented. Many other types of data processing systems may be likewise configured to specifically implement the mechanisms of the illustrative embodiments.

FIG. 6 is a block diagram of an example data processing system in which aspects of the illustrative embodiments are implemented. Data processing system 600 is an example of a computer, such as server 504A or client 510 in FIG. 5, in which computer usable code or instructions implementing the processes for illustrative embodiments of the present invention are located. In one illustrative embodiment, FIG. 6 represents a server computing device, such as a server 504, which implements a cognitive computing system 500 and QA system pipeline 508 of FIG. 5, augmented to include the additional mechanisms of the illustrative embodiments described hereafter.

In the depicted example, data processing system 600 employs a hub architecture including North Bridge and Memory Controller Hub (NB/MCH) 602 and South Bridge and Input/Output (I/O) Controller Hub (SB/ICH) 604. Processing unit 606, main memory 608, and graphics processor 610 are connected to NB/MCH 602. Graphics processor 610 is connected to NB/MCH 602 through an accelerated graphics port (AGP).

In the depicted example, local area network (LAN) adapter 612 connects to SB/ICH 604. Audio adapter 616, keyboard and mouse adapter 620, modem 622, read only memory (ROM) 624, hard disk drive (HDD) 626, CD-ROM drive 630, universal serial bus (USB) ports and other communication ports 632, and PCI/PCIe devices 634 connect to SB/ICH 604 through bus 638 and bus 640. PCI/PCIe devices may include, for example, Ethernet adapters, add-in cards, and PC cards for notebook computers. PCI uses a card bus controller, while PCIe does not. ROM 624 may be, for example, a flash basic input/output system (BIOS).

HDD 626 and CD-ROM drive 630 connect to SB/ICH 604 through bus 640. HDD 626 and CD-ROM drive 630 may use, for example, an integrated drive electronics (IDE) or serial advanced technology attachment (SATA) interface. Super I/O (SIO) device 636 is connected to SB/ICH 604.

An operating system runs on processing unit 606. The operating system coordinates and provides control of various components within the data processing system 600 in FIG. 6. As a client, the operating system is a commercially available operating system such as Microsoft® Windows 10®. An object-oriented programming system, such as the Java™ programming system, may run in conjunction with the operating system and provides calls to the operating system from Java™ programs or applications executing on data processing system 600.

As a server, data processing system 600 may be, for example, an IBM® eServer™ System P® computer system, running the Advanced Interactive Executive (AIX®) operating system or the LINUX® operating system. Data processing system 600 may be a symmetric multiprocessor (SMP) system including a plurality of processors in processing unit 606. Alternatively, a single processor system may be employed.

Instructions for the operating system, the object-oriented programming system, and applications or programs are located on storage devices, such as HDD 626, and are loaded into main memory 608 for execution by processing unit 606. The processes for illustrative embodiments of the present invention are performed by processing unit 606 using computer usable program code, which is located in a memory such as, for example, main memory 608, ROM 624, or in one or more peripheral devices 626 and 630, for example.

A bus system, such as bus 638 or bus 640 as shown in FIG. 6, is comprised of one or more buses. Of course, the bus system may be implemented using any type of communication fabric or architecture that provides for a transfer of data between different components or devices attached to the fabric or architecture. A communication unit, such as modem 622 or network adapter 612 of FIG. 6, includes one or more devices used to transmit and receive data. A memory may be, for example, main memory 608, ROM 624, or a cache such as found in NB/MCH 602 in FIG. 6.

Those of ordinary skill in the art will appreciate that the hardware depicted in FIGS. 5 and 6 may vary depending on the implementation. Other internal hardware or peripheral devices, such as flash memory, equivalent non-volatile memory, or optical disk drives and the like, may be used in addition to or in place of the hardware depicted in FIGS. 5 and 6. Also, the processes of the illustrative embodiments may be applied to a multiprocessor data processing system, other than the SMP system mentioned previously, without departing from the spirit and scope of the present invention.

Moreover, the data processing system 600 may take the form of any of a number of different data processing systems including client computing devices, server computing devices, a tablet computer, laptop computer, telephone or other communication device, a personal digital assistant (PDA), or the like. In some illustrative examples, data processing system 300 may be a portable computing device that is configured with flash memory to provide non-volatile memory for storing operating system files and/or user-generated data, for example. Essentially, data processing system 600 may be any known or later developed data processing system without architectural limitation.

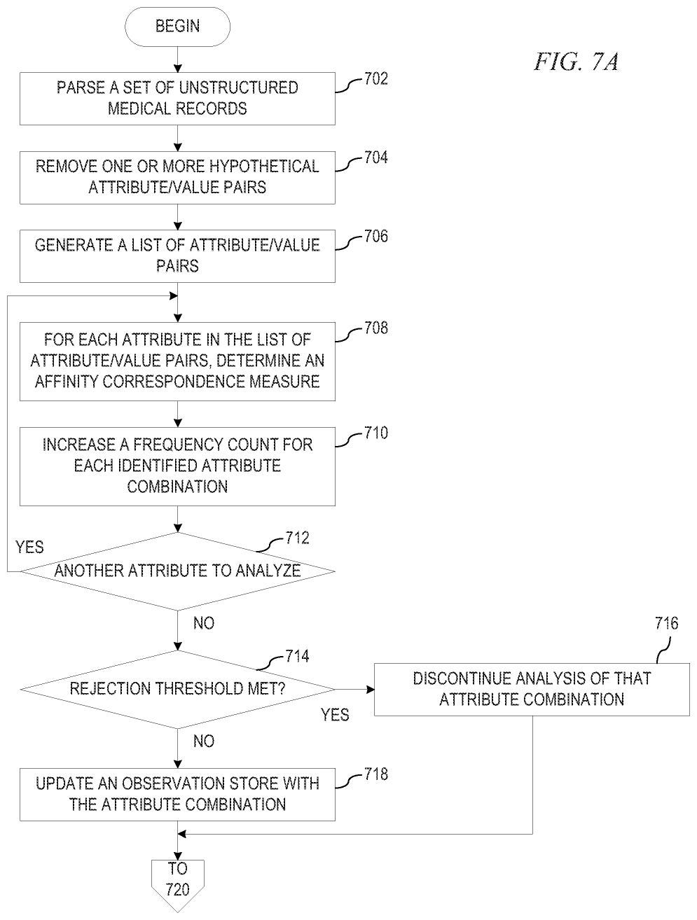

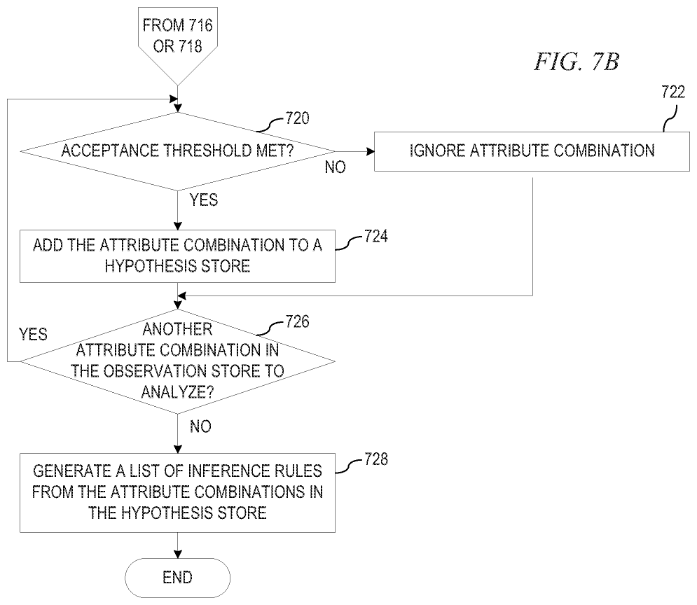

FIGS. 7A and 7B depict an exemplary flowchart outlining example operations performed by a cognitive computing system implementing a clinical inference rules identification mechanism for automatically identifying clinical inference rules in accordance with one illustrative embodiment. As the exemplary operation begins, the clinical inference rules identification mechanism parses a set of unstructured medical records (natural language documents) in a corpus using a set of NLP machine learning and/or rule techniques and predefined attributes in NLP machine learning and/or rule techniques and predefined attributes thereby generating a set of attributes and corresponding values thereby forming a set of attribute/value pairs (step 702). The clinical inference rules identification mechanism then removes one or more attribute/value pairs in the set of attribute/value pairs identified as being hypothetical (step 704). The clinical inference rules identification mechanism then generates a list of attribute/value pairs (step 706).

For each attribute in the list of attribute/value pairs, the clinical inference rules identification mechanism determines an affinity correspondence measure of the attribute/value pair with each other attribute/value pair in the set of attribute/value pairs (step 708). For each identified combination, the clinical inference rules identification mechanism increases a frequency count for the combination (step 710). The clinical inference rules identification mechanism then determines whether there is another attribute to analyze (step 712). If at step 712 there is another attribute to analyze, then the operation returns to step 708. If at step 712 there is not another attribute to analyze, then the clinical inference rules identification mechanism determines whether a rejection threshold has been met for the attribute combination, such as, for example, a frequency of the combination is >0.05 which means high variance and/or no correlation (step 714). If at step 714 the clinical inference rules identification mechanism determines that the rejection threshold has been met, then the clinical inference rules identification mechanism discontinues analysis of that attribute combination for any further attribute sets of additional patients and moves the attribute combination to a coincidence list (step 716). If at step 714 the clinical inference rules identification mechanism determines that the rejection threshold has not been met, then the clinical inference rules identification mechanism updates an observation store with the attribute combination (step 718). By clinical inference rules identification mechanism removing attribute pair combinations based on the rejection threshold, the clinical inference rules identification mechanism reduces a number of attribute pairs under consideration, which decreases computational processing time.

For each remaining attribute combination in the observation store, the clinical inference rules identification mechanism determines whether the attribute combination meets an acceptance threshold indicating low variance and/or 95% correspondence (step 720). If at step 720 the clinical inference rules identification mechanism determines that the attribute combination fails to meet the acceptance threshold, then the clinical inference rules identification mechanism ignores the attribute combination (step 722). If at step 720 the clinical inference rules identification mechanism determines that attribute combination meets the acceptance threshold, then the clinical inference rules identification mechanism adds the attribute combination to a hypothesis store (step 724). From step 722 or step 724, the clinical inference rules identification mechanism determines whether there is another attribute combination in the observation store to analyze (step 726). If at step 726 the clinical inference rules identification mechanism determines that there is another attribute combination in the observation store to analyze, the operation returns to step 720. If at step 726 the clinical inference rules identification mechanism determines that there is no other attribute combination in the observation store to analyze, the clinical inference rules identification mechanism generates a list of inference rules from the attribute combinations in the hypothesis store (step 728), with the operation terminating thereafter.

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or IO devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication based network adapters may also be utilized including, but not limited to, 802.11 a/b/g/n wireless communication adapters, Bluetooth wireless adapters, and the like. Any known or later developed network adapters are intended to be within the spirit and scope of the present invention.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, in a cognitive computing system comprising at least one processor and at least one memory, wherein the at least one memory comprises instructions that are executed by the at least one processor to configure the at least one processor to implement an inference rules identification mechanism for automatically identifying inference rules, the method comprising:
parsing a content of at least one natural language document in a collection of natural language documents utilizing natural language processing to identify a set of attributes and corresponding values present in the content of the at least one natural language document thereby forming a set of attribute/value pairs;
for each attribute/value pair in the set of attribute/value pairs, determining an affinity correspondence measure of the attribute/value pair with each other attribute/value pair in the set of attribute/value pairs, wherein the affinity correspondence measure indicates an affinity of one attribute/value pair to another attribute/value pair in the set of attribute/value pairs;
determining, based on the affinity correspondence measures of each attribute/value pair with each other attribute/value pair in the set of attribute/value pairs, a set of inferred rules, wherein each inferred rule in the set of inferred rules indicates a relationship between the attribute/value pair and a corresponding attribute/value pair, and
automatically generating the inferred rules as rule data structures that are implemented in a cognitive computing model of the cognitive computing system.

2. The method of claim 1, wherein the affinity correspondence measure is generated across multiple sets of attribute/value pair obtained from multiple different natural language documents.

3. The method of claim 1, wherein the at least one natural language document comprises electronic medical record documents associated with a set of patients and wherein each set of attribute/value pairs is associated with a different patient in the set of patients.

4. The method of claim 1, further comprising:
performing natural language processing on the collection of natural language documents to identify instances of attribute/value pairs in the set of attribute/value pairs corresponding to hypothetical natural language content, wherein the identified instances are hypothetical attribute/value pairs; and
removing the hypothetical attribute/value pairs from the set of attribute/value pairs prior to the determining and automatically generating operations.

5. The method of claim 1, wherein determining the affinity correspondence measure comprises:
counting a number of instances of each attribute or the attribute/value pairs in the set of attribute/value pairs in the collection of natural language documents;
for each first attribute/value pair in the set of attribute/value pairs, comparing the first attribute/value pair to each other second attribute/value pair in the set of attribute/value pairs and determining a frequency count of a number of instances of co-occurrence of the first attribute/value pair with each other second attribute/value pairs on documents of the collection of natural language documents;
in response to the frequency count being above a rejection threshold, removing the combination of the first attribute/value pair and the second attribute/value pair from further processing; and
generating an attribute/value pair hypothesis data structure specifying first attribute/value pairs and second attribute/value pairs that have statistically significant correlations with one another based on the frequency count being equal to or below the rejection threshold.

6. The method of claim 5, wherein the inferred rules are generated based on correlations of first attribute/value pairs and second attribute/value pairs specified in the attribute/value pair hypothesis data structure.

7. The method of claim 1, where in the inferred rules that are automatically generated as rule data structures implemented in the cognitive computing model of the cognitive computing system are used process other natural language documents.

8. A computer program product comprising a computer readable storage medium having a computer readable program stored therein, wherein the computer readable program, when executed on a data processing system, causes the data processing system to implement an inference rules identification cognitive computing system for automatically identifying inference rules, and further causes the data processing system to:
parse a content of at least one natural language document in a collection of natural language documents utilizing natural language processing to identify a set of attributes and corresponding values present in the content of the at least one natural language document thereby forming a set of attribute/value pairs;
for each attribute/value pair in the set of attribute/value pairs, determine an affinity correspondence measure of the attribute/value pair with each other attribute/value pair in the set of attribute/value pairs, wherein the affinity correspondence measure indicates an affinity of one attribute/value pair to another attribute/value pair in the set of attribute/value pairs;
determine, based on the affinity correspondence measures of each attribute/value pair with each other attribute/value pair in the set of attribute/value pairs, a set of inferred rules, wherein each inferred rule in the set of inferred rules indicates a relationship between the attribute/value pair and a corresponding attribute/value pair; and
automatically generate the inferred rules as rule data structures that are implemented in a cognitive computing model of the cognitive computing system.

9. The computer program product of claim 8, wherein the affinity correspondence measure is generated across multiple sets of attribute/value pair obtained from multiple different natural language documents.

10. The computer program product of claim 8, wherein the at least one natural language document comprises electronic medical record documents associated with a set of patients and wherein each set of attribute/value pairs is associated with a different patient in the set of patients.

11. The computer program product of claim 8, wherein the computer readable program further causes the data processing system to:
perform natural language processing on the collection of natural language documents to identify instances of attribute/value pairs in the set of attribute/value pairs corresponding to hypothetical natural language content, wherein the identified instances are hypothetical attribute/value pairs; and
remove the hypothetical attribute/value pairs from the set of attribute/value pairs prior to the determining and automatically generating operations.

12. The computer program product of claim 8, wherein the computer readable program to determine the affinity correspondence measure further causes the data processing system to:
count a number of instances of each attribute or the attribute/value pairs in the set of attribute/value pairs in the collection of natural language documents;
for each first attribute/value pair in the set of attribute/value pairs, compare the first attribute/value pair to each other second attribute/value pair in the set of attribute/value pairs and determining a frequency count of a number of instances of co-occurrence of the first attribute/value pair with each other second attribute/value pairs on documents of the collection of natural language documents;
in response to the frequency count being above a rejection threshold, remove the combination of the first attribute/value pair and the second attribute/value pair from further processing; and
generate an attribute/value pair hypothesis data structure specifying first attribute/value pairs and second attribute/value pairs that have statistically significant correlations with one another based on the frequency count being equal to or below the rejection threshold.

13. The computer program product of claim 12, wherein the inferred rules are generated based on correlations of first attribute/value pairs and second attribute/value pairs specified in the attribute/value pair hypothesis data structure.

14. The computer program product of claim 8, where in the inferred rules that are automatically generated as rule data structures implemented in the cognitive computing model of the cognitive computing system are used process other natural language documents.

15. A data processing system comprising:
at least one processor; and
at least one memory coupled to the at least one processor, wherein the at least one memory comprises instructions which, when executed by the at least one processor, cause the at least one processor to implement an inference rules identification cognitive computing system for automatically identifying inference rules, and further cause the at least one processor to:
parse a content of at least one natural language document in a collection of natural language documents utilizing natural language processing to identify a set of attributes and corresponding values present in the content of the at least one natural language document thereby forming a set of attribute/value pairs;
for each attribute/value pair in the set of attribute/value pairs, determine an affinity correspondence measure of the attribute/value pair with each other attribute/value pair in the set of attribute/value pairs, wherein the affinity correspondence measure indicates an affinity of one attribute/value pair to another attribute/value pair in the set of attribute/value pairs;
determine, based on the affinity correspondence measures of each attribute/value pair with each other attribute/value pair in the set of attribute/value pairs, a set of inferred rules, wherein each inferred rule in the set of inferred rules indicates a relationship between the attribute/value pair and a corresponding attribute/value pair, and
automatically generate the inferred rules as rule data structures that are implemented in a cognitive computing model of the cognitive computing system.

16. The data processing system of claim 15, wherein the affinity correspondence measure is generated across multiple sets of attribute/value pair obtained from multiple different natural language documents.

17. The data processing system of claim 15, wherein the at least one natural language document comprises electronic medical record documents associated with a set of patients and wherein each set of attribute/value pairs is associated with a different patient in the set of patients.

18. The data processing system of claim 15, wherein the instructions further cause the processor to:
perform natural language processing on the collection of natural language documents to identify instances of attribute/value pairs in the set of attribute/value pairs corresponding to hypothetical natural language content, wherein the identified instances are hypothetical attribute/value pairs; and
remove the hypothetical attribute/value pairs from the set of attribute/value pairs prior to the determining and automatically generating operations.

19. The data processing system of claim 15, wherein the instructions to determine the affinity correspondence measure further cause the processor to:
count a number of instances of each attribute or the attribute/value pairs in the set of attribute/value pairs in the collection of natural language documents;
for each first attribute/value pair in the set of attribute/value pairs, compare the first attribute/value pair to each other second attribute/value pair in the set of attribute/value pairs and determining a frequency count of a number of instances of co-occurrence of the first attribute/value pair with each other second attribute/value pairs on documents of the collection of natural language documents;
in response to the frequency count being above a rejection threshold, remove the combination of the first attribute/value pair and the second attribute/value pair from further processing; and
generate an attribute/value pair hypothesis data structure specifying first attribute/value pairs and second attribute/value pairs that have statistically significant correlations with one another based on the frequency count being equal to or below the rejection threshold.

20. The data processing system of claim 19, wherein the inferred rules are generated based on correlations of first attribute/value pairs and second attribute/value pairs specified in the attribute/value pair hypothesis data structure.
